(19) United States
(12) Patent Application Publication
(10) Pub. No.: US 2026/0272938 A1
(43) Pub. Date: Sep. 17, 2026

(54) USE OF TOPICAL BRAF INHIBITOR COMPOSITIONS FOR TREATMENT OF HISTIOCYTOSIS

(71) Applicant: Lutris Pharma Ltd., Tel Aviv (IL)

(72) Inventor: Noa Shelach, Tel Aviv (IL)

(21) Appl. No.: 19/563,681

(22) Filed: Mar. 11, 2026

Related U.S. Application Data
(60) Provisional application No. 63/770,098, filed on Mar. 11, 2025.

(51) Int. Cl. A61K 31/52; A61K 9/00; A61K 31/437; A61K 31/506; A61K 31/517; A61P 17/00; A61P 35/00

(52) U.S. Cl. CPC A61K 31/52; A61K 9/0014; A61K 31/437; A61K 31/506; A61K 31/517; A61P 17/00; A61P 35/00

(57) ABSTRACT

Provided are pharmaceutical topical compositions, comprising a therapeutically-effective amount of at least one compound of Formula I-IV and methods of using pharmaceutical topical compositions comprising a compound of Formula I-IV for the treatment, prevention of histiocytosis escalation and reduction of the severity of histiocytosis.

### CROSS-REFERENCE TO COPENDING APPLICATION

This application claims priority to U.S. Provisional Patent Application 63/770,098 filed on Mar. 11, 2025, entitled “Use of BRAF Compositions for Treatment of Histiocytosis,” the contents of which are all herein incorporated by reference.

### FIELD OF THE INVENTION

The present disclosure relates to compositions and methods of using topical BRaf inhibitor compositions for treatment of histiocytosis.

### BACKGROUND OF THE INVENTION

Histiocytosis is a group of rare diseases sharing a common characteristic—an excessive number of histiocytes (tissue macrophages), (eMedicine Dictionary).

### SUMMARY OF THE INVENTION

Topical compositions comprising at least one compound of Formula I-IV of this disclosure and methods of using a composition comprising at least one compound of Formula I-IV for the treatment, prevention of histiocytosis escalation and/or reduction of histiocytosis severity are provided.

Also provided are pharmaceutical topical compositions comprising at least one compound of Formula I-IV (e.g. Formula IV) and method of using pharmaceutical topical compositions comprising compound of Formula I-IV for the treatment, prevention of histiocytosis escalation and/or reduction of histiocytosis severity.

The drug products comprising a compound of Formula I-IV (alone or in combination with vemurafenib or other approvable therapeutically effective drug or treatment) are useful in treating several types of histiocytosis, whether topical, systemic or both.

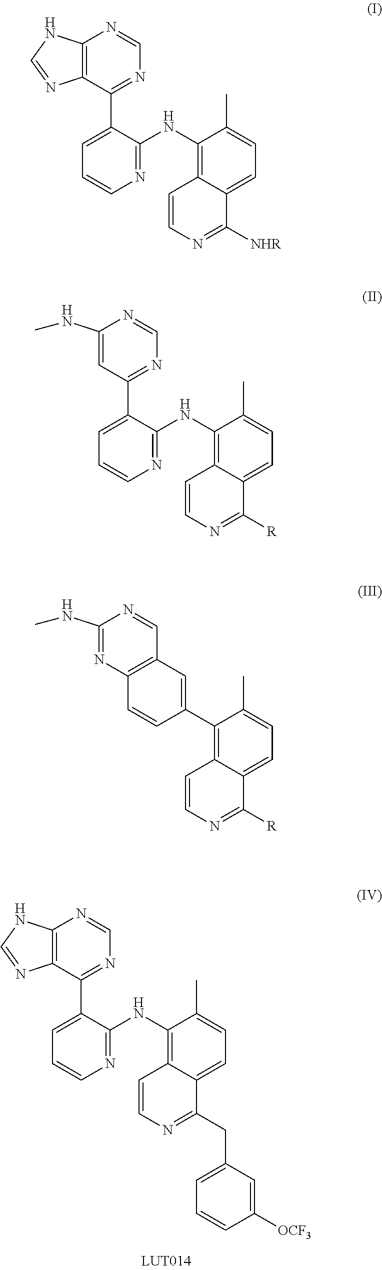

Without wishing to be bound by theory, the mechanism involved may be anti-proliferative.

### Target Populations:

1. Cutaneous histiocytosis subjects having only histiocytosis skin lesions. These subjects are treated only with a topical composition comprising at least one compound of Formula I-IV of this disclosure.
2. Histiocytosis subjects having also histiocytosis skin lesions (usually under the eyes)

These subjects are treated concomitantly with a therapeutically effective amount of oral vemurafenib or any other effective BRaf inhibitor or treatment and a topical composition comprising a therapeutically effective amount of at least one compound of Formula I-IV of this disclosure.

In some aspects, the above topical compositions comprise from 0.01% to 1.00% weight/weight of at least one compound of Formula I-IV.

In some aspects, the topical composition comprises about 0.03% weight/weight of the compound of Formula I-IV.

In some aspects, the topical composition comprises about 0.03% weight/weight of the compound of Formula IV.

In some aspects, the topical pharmaceutical composition is in the form of a gel, a hydrogel, an ointment, a cream, a spray, a dermal patch, a foam, a lotion, a solution or a suspension. In some embodiments, the topical pharmaceutical composition is in the form of a gel. In some embodiments, the topical pharmaceutical composition is in the form of a spray.

Vemurafenib exhibited dramatic efficacy in the treatment of two types of histiocytosis (“Dramatic efficacy of vemurafenib in both multisystemic and refractory Erdheim-Chester disease and Langerhans cell histiocytosis harboring the BRAF V600E mutation” Julien Haroche et al., pre-published online Dec. 20 2012). DOI: 10.1182/blood-2012-07-446286).

However, the use of oral vemurafenib presents several drawbacks:
a. In subjects having cutaneous lesions the oral vemurafenib treatment is not effective enough in treating cutaneous lesions, and
b. The oral systemic vemurafenib treatment is liable to cause adverse reactions, which are deleterious to optimal dosing and patient compliance.

In the context of this disclosure, the term “vemurafenib” is used interchangeably with “Zelboraf” or “Zelboraf™”, an FDA-approved BRaf inhibitor for treatment of late-stage melanoma. Zelboraf is systemically (orally) administered.

In some aspects, this disclosure provides a method for treatment of cutaneous histiocytosis by topical administration of a composition comprising at least one compound of Formula I-IV on the cutaneous lesions of a histiocytosis subject in need thereof.

In some aspects, this disclosure provides a method for treatment of cutaneous histiocytosis alone lesions), by concomitant administration of only one product to a cutaneous histiocytosis subject, the product comprising a therapeutically effective amount of a compound of Formula I-IV administered topically to the skin of said subject.

In some aspects, this disclosure provides a method for treatment of histiocytosis accompanied with cutaneous lesions), by concomitant administration of two drug products to a histiocytosis subject.
1. A topical product comprising at least one compound of Formula I-IV administered topically to the skin of said subject.
2. An oral product or treatment comprising a therapeutically effective dose of a drug or treatment (e.g. vemurafenib).

In some aspects, the two drug products may be prescribed and distributed separately or alternatively as one product formulated with fixed doses of the two drugs or treatments or as a kit of the two products.

In some aspects, the method reduces the severity or prevents escalation of the histiocytosis cutaneous lesions.

In some aspects, the method reduces the severity of the histiocytosis accompanied with topical lesions

Aspects of this disclosure relate to a topical pharmaceutical composition comprising from 0.01% weight/weight to 0.3% weight/weight of a compound of Formula IV and a pharmaceutically acceptable carrier or excipient.

In some aspects, the composition is in the form of a gel, a hydrogel, an ointment, a cream, a foam, a spray, a lotion, a liquid or a dermal patch.

There is still a need in the art for the development of novel therapeutic compounds, compositions, and methods of treatment, prevention of escalation and reduction of the severity of the cutaneous histiocytosis.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

According to the Histiocytosis Association, 1 in 200,000 children in the United States are born with histiocytosis each year. HAA also states that most of the people diagnosed with histiocytosis are children under the age of 10, although the disease can afflict adults. The disease usually occurs from birth to age 15.

The group of histiocytosis diseases is classified in various ways.

According to the 1999 classification proposed by the World Health Organization, they can be divided into three categories, as shown below (Wikipedia monograph):

### Classifications

| Name | WHO | ICD10 | MeSH |
|---|---|---|---|
| Langerhans cell | I | D76.0 | Langerhans-cell histiocytosis |
| histiocytosis (LCH) | | | |
| Juvenile | II | D76.3 | non-Langerhans-cell |
| xanthogranuloma (JXG) | | | histiocytosis |
| Hemophagocytic | II | D76.1 | non-Langerhans-cell |
| lymphohistiocytosis | | | histiocytosis |
| Niemann-Pick disease | II | E75.2 | non-Langerhans-cell |
| | | | histiocytosis |
| Sea-blue histiocytosis | II | — | non-Langerhans-cell |
| | | | histiocytosis |
| Acute monocytic | III | C93.0 | malignant histiocytic disorders |
| leukemia | | | |
| Malignant histiocytosis | III | C96.1 | malignant histiocytic disorders |
| Erdheim-Chester disease | II | C96.1 | malignant histiocytic disorders |

Alternatively, histiocytoses may be divided into the following groups:
X-type histiocytoses
Non-X histiocytoses

Oral Vemurafenib (Zelboraf™), a BRaf inhibitor, has proved itself very effective in treatment of histiocytoses.

### Definitions

Unless defined otherwise, all technical and scientific terms used herein have the same meaning as commonly understood to one of ordinary skill in the art to which this disclosure pertains. Otherwise, certain terms used herein have the meanings as set forth in the specification. All patents, published patent applications and publications cited herein are incorporated by reference as if set forth fully herein. It must be noted that as used herein and in the appended claims, the singular forms “a,” “an,” and “the” include plural references unless the context clearly dictates otherwise.

The term “about” when used before a numerical designation, e.g., temperature, time, amount, and concentration, including range, indicates approximations which may vary by (+) or (−) 10%, 5% or 1%.

The terms “pharmaceutically acceptable salts” or “salts thereof” mean salts which are pharmaceutically acceptable, and which possess the desired pharmacological activity.

The terms “treat,” “treating,” or “treatment,” as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting or suppressing the development of the disease or condition, relieving the disease or condition, causing regression of the disease or condition, relieving a condition caused by the disease or condition, or suppressing the symptoms of the disease or condition, and are intended to include prophylaxis. The terms also include relieving the disease or conditions, e.g., causing the regression of clinical symptoms. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The term “carrier” as used herein, refers to nontoxic chemical compounds or agents that facilitate the incorporation of a compound into cells, e.g., dermal cells, or tissues. Carriers useful herein include any such materials known in the art, which are nontoxic and do not interact with other components of the formulation in which it is contained in a deleterious manner. As used herein, “pharmaceutically acceptable carrier” includes any and all solvents, dispersion media, and the like, which are pharmaceutically acceptable.

The term “therapeutically effective amount” of a compound or a composition refers to that amount of the compound or the composition that results in treatment, including reduction or inhibition of symptoms in a patient.

The term “subject” as used herein refers to organisms to be treated by the compounds or compositions of the present disclosure. Such organisms include a mammal, including a human or non-human mammal. The terms patient, individual and subject may be used interchangeably.

BRaf is a protein kinase involved in the regulation of the mitogen activated protein kinase. Histiocytosis is a proliferation of Langerhans cells, in which BRAF mutations, usually V600E, have been reported in 40%-70% of cases. Among patients with BRAFV600E-mutated histiocytosis, targeted therapy with BRAF inhibitors represents a novel therapeutic approach.

Provided herein are the compounds of Formula I-IV and compositions comprising them.

### Compounds

In one embodiment, provided herein is a compound of Formula (I):

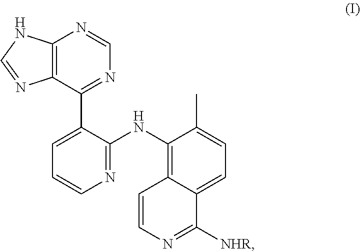

wherein R is selected from the group consisting of 3-ethynylphenyl (code name LUT012), 3-chloro-4-fluorophenyl (LUT015), 2-fluoro-4-iodophenyl (LUT016), 4-chloro-3-(trifluoromethyl)phenyl (LUT017), 3-(1,1-dimethylethyl)-1-methyl-1H-pyrazol-5-yl (LUT013), 3-(trifluoromethoxy)phenyl (LUT014), or a pharmaceutically acceptable salt or a solvate thereof.

In another embodiment, provided herein is a compound of Formula (II):

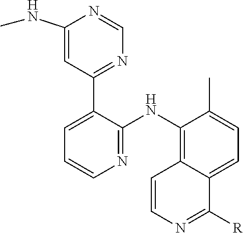

wherein R is NHR¹, wherein R¹ is 2-fluoro-4-iodophenyl, or a pharmaceutically acceptable salt or a solvate thereof.

In another embodiment, provided herein is a compound of Formula (III):

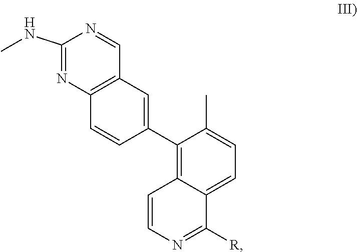

wherein R is NHR¹, wherein R¹ is 3-ethynylphenyl, 3-chloro-4-fluorophenyl, 2-fluoro-4-iodophenyl, or 4-chloro-3-(trifluoromethyl)phenyl, or a pharmaceutically acceptable salt or a solvate thereof.

In one embodiment, provided herein is a compound of Formula (IV)

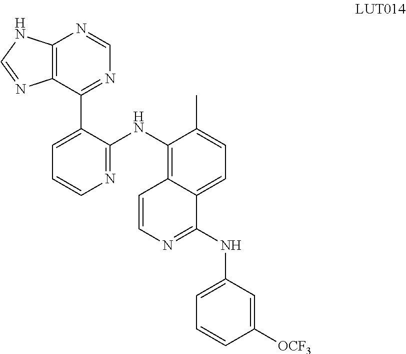 [SMILES: Cc1ccc2c(Nc3cccc(OC(F)(F)F)c3)nccc2c1Nc1ncccc1-c1ncnc2[nH]cnc12]

Compound of Formula IV (code name LUT014) is part of the Formula I family of compounds, wherein R is 3-(trifluoromethoxy)phenyl and was selected in the first screening for further development.

In one embodiment, the compounds of present disclosure inhibit the activity of BRaf. In one embodiment, the compounds of present disclosure may have an IC50 towards Braf of about 0.5×10⁻⁸ M to about 5×10⁻⁸ M, about 1×10⁻⁸ M to about 5×10⁻⁸ M, about 1×10⁻⁸ M to about 3.5×10⁻⁸ M, or about 1×10⁻⁸ M to about 3×10⁻⁸ M.

In one embodiment, the compounds of present disclosure show no phototoxicity or show reduced phototoxicity. The level of phototoxicity can be determined by measuring a Photo-Irritation Factor (PIF) or a Mean Photo Effect (MPE).
In one embodiment, a PIF can be calculated using the following formula: PIF=IC50(−Irr)/IC50(+Irr), where PIF>5 indicates phototoxicity; 2<PIF<5 indicates probable phototoxicity; PIF<2 indicates no phototoxicity. In one embodiment, the compounds of present disclosure have a PIF of less than 5. In another embodiment, the compounds of present disclosure have a PIF of less than 2.

In one embodiment, the MPE can be calculated by comparing the complete concentration-response curves. MPE is a weighted average of the difference in response of equivalent doses normalized by the shift in IC50. MPE>0.15 indicates phototoxicity; 0.1<MPE<0.15 indicates probable phototoxicity; MPE<0.1 indicates no phototoxicity. In one embodiment, the compounds of present disclosure have a MPE of less than 0.15. In another embodiment, the compounds of present disclosure have a MPE of less than 0.1.

### Compositions

In some embodiments, provided herein are pharmaceutical compositions comprising a compound of Formula (I), (II) or (III) or a pharmaceutically acceptable salt or a solvate thereof, or a combination thereof, and a pharmaceutically acceptable carrier or excipient.

In some embodiments, provided herein are pharmaceutical compositions comprising a compound of Formula (IV) or a pharmaceutically acceptable salt or a solvate thereof, or a combination thereof, and a pharmaceutically acceptable carrier or excipient.

In some embodiments, the pharmaceutical composition comprises about 0.01% w/w to about 1% w/w of the compound or a pharmaceutically acceptable salt or a solvate thereof, or a combination thereof, based on the total weight of the composition. In some embodiments, the pharmaceutical composition comprises about 0.01% w/w to about 0.1% w/w of the compound or a pharmaceutically acceptable salt or a solvate thereof, or a combination thereof, based on the total weight of the composition. For example, the pharmaceutical topical composition comprises about 0.01, 0.02, 0.03, 0.04, 0.05, 0.06, 0.07, 0.08, 0.09, 0.1% w/w, including values and ranges therebetween, of any of the compounds disclosed herein or combinations thereof. In some embodiments, the pharmaceutical topical composition comprises from about 0.01% to about 0.03%, about 0.01% to about 0.04%, about 0.01% to about 0.05%, about 0.01% to about 0.06%, about 0.01% to 0.07%, about 0.01% to about 0.08%, about 0.01% to about 0.09%, about 0.01% to about 0.1%, 0.02% to about 0.03%, about 0.02% to about 0.04%, about 0.02% to about 0.05%, about 0.02% to about 0.06%, about 0.02% to 0.07%, about 0.02% to about 0.08%, about 0.02% to about 0.02%, about 0.02% to about 0.1%, 0.03% to about 0.04%, about 0.03% to about 0.05%, about 0.03% to about 0.06%, about 0.03% to 0.07%, about 0.03% to about 0.08%, about 0.03% to about 0.09%, about 0.03% to about 0.1%, about 0.04% to about 0.05%, about 0.04% to about 0.06%, about 0.04% to 0.07%, about 0.04% to about 0.08%, about 0.04% to about 0.09%, about 0.04% to about 0.1%, %, about 0.05% to about 0.06%, about 0.05% to 0.07%, about 0.05% to about 0.08%, about 0.05% to about 0.09%, about 0.05% to about 0.1% weight/weight (w/w), including values and ranges therebetween, of any of the compounds disclosed herein.

In some embodiments, the pharmaceutical composition comprises about 0.03% w/w to about 0.25% w/w of the compound or a pharmaceutically acceptable salt or a solvate thereof, or a combination thereof, based on the total weight of the composition. For example, the pharmaceutical composition can comprise about 0.03, 0.04, 0.05, 0.06, 0.07, 0.08, 0.09, 0.1, 0.11, 0.12, 0.13, 01.4, 0.15, 0.16, 0.17, 0.18, 0.19, 0.20, 0.21, 0.22, 15 0.23, 0.24, 0.25% w/w, including values and ranges therebetween, of any of the compounds disclosed herein or combinations thereof. In some embodiments, the pharmaceutical composition comprises from about 0.03% to about 0.04%, about 0.03% to about 0.05%, about 0.03% to about 0.06%, about 0.03% to 0.07%, about 0.03% to about 0.08%, about 0.03% to about 0.09%, about 0.03% to about 0.1%, about 0.03% to 20 about 0.11%, about 0.03% to about 0.12%, about 0.03% to about 0.13%, about 0.03% to about 0.14%, about 0.03% to about 0.15%, about 0.03% to about 0.16%, about 0.03% to about 0.17%, about 0.03% to about 0.18%, about 0.03% to about 0.19%, about 0.03% to about 0.2%, about 0.03% to about 0.21%, about 0.03% to about 0.22%, about 0.03% to about 0.23%, about 0.03% to about 0.24%, about 0.03% to about 0.25 weight/weight 25 (w/w), including values and ranges therebetween, of any of the compounds disclosed herein.

In some embodiments, the pharmaceutical composition comprises about 0.01% w/w to about 0.1% w/w of the compound of Formula (IV) or a pharmaceutically acceptable salt or a solvate thereof, or a combination thereof, based on the total weight of the composition. For example, the pharmaceutical composition may comprise about 0.01, 0.02, 0.03, 0.04, 0.05, 0.06, 0.07, 0.08, 0.09, 0.1% w/w, including values and ranges therebetween, of any of the compounds disclosed herein or combinations thereof. In some embodiments, the pharmaceutical composition comprises from about 0.01% to about 0.03%, about 0.01% to about 0.04%, about 0.01% to about 0.05%, about 0.01% to about 0.06%, about 0.01% to 0.07%, about 0.01% to about 0.08%, about 0.01% to about 0.09%, about 0.01% to about 0.1%, 0.02% to about 0.03%, about 0.02% to about 0.04%, about 0.02% to about 0.05%, about 0.02% to about 0.06%, about 0.02% to 0.07%, about 0.02% to about 0.08%, about 0.02% to about 0.02%, about 0.02% to about 0.1%, 0.03% to about 0.04%, about 0.03% to about 0.05%, about 0.03% to about 0.06%, about 0.03% to 0.07%, about 0.03% to about 0.08%, about 0.03% to about 0.09%, about % to about 0.1%, about 0.04% to about 0.05%, about 0.04% to about 0.06%, about % to 0.07%, about 0.04% to about 0.08%, about 0.04% to about 0.09%, about 0.04% to about 0.1%, %, about 0.05% to about 0.06%, about 0.05% to 0.07%, about 0.05% to about 0.08%, about 0.05% to about 0.09%, about 0.05% to about 0.1% w/w, including values and ranges therebetween, of any of the compounds disclosed herein.

In some embodiments, the pharmaceutical composition comprises about 0.03% w/w to about 0.1% w/w of the compound of Formula (IV) or a pharmaceutically acceptable salt or a solvate thereof, or a combination thereof, based on the total weight of the composition. For example, the pharmaceutical composition may comprise about 0.01, 0.02, 0.03, 0.04, 0.05, 0.06, 0.07, 0.08, 0.09, 0.1% w/w, including values and ranges therebetween, of any of the compounds disclosed herein or combinations thereof. In some embodiments, the pharmaceutical composition comprises from about 0.03% to about 0.04%, about 0.03% to about 0.05%, about 0.03% to about 0.06%, about 0.03% to 0.07%, about 0.03% to about 0.08%, about 0.03% to about 0.09%, about 0.03% to about 0.1%, about 0.04% to about 0.05%, about 0.04% to about 0.06%, about 0.04% to 0.07%, about 0.04% to about 0.08%, about 0.04% to about 0.09%, about 0.04% to about 0.1%, about 0.05% to about 0.06%, about 0.05% to 0.07%, about 0.05% to about 0.08%, about 0.05% to about 0.09%, about 0.05% to about 0.1% w/w, including values and ranges therebetween, of any of the compounds disclosed herein.

In some embodiments, the pharmaceutical composition comprising any one of the compounds disclosed herein is formulated for topical administration.

In some embodiments, provided herein is a topical pharmaceutical composition comprising a compound of Formula (I-IV), a pharmaceutically acceptable salt or a solvate thereof, or a combination thereof, and a pharmaceutically acceptable carrier or excipient. Compositions for topical administration (topical compositions) can be formulated in the form of a gel, a hydrogel, an ointment, a cream, a paste, a foam, a spray, a lotion, a liquid, or a dermal patch and may comprise any of the disclosed compound(s) in any of the amounts described herein.

In some embodiments, provided herein is a topical pharmaceutical composition comprising a compound of Formula (IV) or a pharmaceutically acceptable salt or a solvate thereof, or a combination thereof, and a pharmaceutically acceptable carrier or excipient.

Compositions for topical administration (topical compositions) can be in the form of a gel, a hydrogel, an ointment, a cream, a paste, a foam, a spray, a lotion, a liquid, or a dermal patch and may comprise any of the disclosed compound(s) in any of the amounts described herein.

In some embodiments, the topical composition is a gel and comprises about 0.01% w/w to about 1% w/w of the compound of Formula I-IV or a pharmaceutically acceptable salt or a solvate thereof, or a combination thereof, and a pharmaceutically acceptable carrier or excipient.

In some embodiments, the topical composition is a gel and comprises about 0.03% w/w to about 0.25% w/w of the compound of Formula (IV) or a pharmaceutically acceptable salt or a solvate thereof, or a combination thereof, and a pharmaceutically acceptable carrier or excipient.

In some embodiments, the topical composition is a gel and comprises about 0.03% w/w to about 0.1% w/w of the compound of Formula (IV) or a pharmaceutically acceptable salt or a solvate thereof, or a combination thereof, and a pharmaceutically acceptable carrier or excipient.

In some embodiments, the topical composition is a gel and comprises about 0.01% w/w to about 0.1% w/w of the compound of Formula (IV) or a pharmaceutically acceptable salt or a solvate thereof, or a combination thereof, and a pharmaceutically acceptable carrier or excipient.

In some embodiments, the topical composition is a gel and comprises about 0.03% w/w of the compound of Formula (IV) or a pharmaceutically acceptable salt or a solvate thereof, or a combination thereof, and a pharmaceutically acceptable carrier or excipient.

Topical compositions useful in the present disclosure may be formulated as a solution. Such compositions may comprise an emollient preferably containing from about 1% to about 50% of an emollient(s). As used herein, the term “emollient” refers to materials used for the prevention or relief of dryness, as well as for the protection of the skin. A number of suitable emollients are known and may be used in the present disclosure. For example, Sagarin, Cosmetics, Science and Technology, 2nd Edition, Vol. 1, pp. 32-43 (1972) and the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Edition, 1997) (hereinafter “ICI Handbook”) contains numerous examples of suitable materials.

A lotion can be made from such a solution. Lotions typically comprise from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically comprises from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may comprise a simple base of animal or vegetable oils or semi-solid hydrocarbons. An ointment may comprise from about 2% to about 10% of an emollient(s) plus from about 0.1% to about 2% of a thickening agent(s). A more complete disclosure of thickening agents or viscosity increasing agents useful herein can be found in Sagarin, Cosmetics, Science and Technology, 2nd Edition, Vol. 1, pp. 72-73 (1972) and the ICI Handbook pp. 1693-1697.

The topical compositions in the present disclosure may be formulated as emulsions. If the carrier for a topical composition is an emulsion, from about 1% to about 10% (e.g., from about 2% to about 5%) of the carrier comprises an emulsifier(s). Emulsifiers may be nonionic, anionic or cationic. Suitable emulsifiers are disclosed in, for example, in Mccutcheon's Detergents and Emulsifiers, North American Edition, pp. 317-324 (1986), and the ICI Handbook, pp. 1673-1686.

Lotions and creams can be formulated as emulsions. Such lotions may comprise from 0.5% to about 5% of an emulsifier(s). Creams may comprise from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

The topical compositions of this disclosure can also be formulated as a gel (e.g., an aqueous, alcohol, alcohol/water, or oil gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils include, but are not limited to, hydrogenated butylene/ethylene/styrene copolymer and hydrogenated ethylene/propylene/styrene copolymer. Gel compositions may comprise between about 0.1% and 5%, by weight, of such gelling agents.

Another topical dosage form is a paste. Pastes are stiff semi-solid topical dosage forms, containing a high proportion of finely powdered solid such as starch, zinc oxide, calcium carbonate and talc. Another component of pastes is a base, which may be a hydrocarbon, an absorption ingredient, a water-miscible ingredient or a water-soluble ingredient. Pastes are less greasy than ointments.

In one embodiment, the topical compositions of this disclosure can also be formulated as a sprayable topical formulation. The compositions can be in a form suitable for application by spraying from an aerosol or pump spray container. The spray formulation can comprise the compound of the disclosure a solution or suspension in a vehicle. The vehicle can optionally contain a polymer or combination of polymers which, when sprayed on the surface of the skin, forms a film on the skin.

In addition to the above carriers and excipients, other emollients and surface active agents can be incorporated into the topical compositions, including glycerol trioleate, acetylated sucrose distearate, sorbitan trioleate, polyoxyethylene (1) monostearate, glycerol monooleate, sucrose distearate, polyethylene glycol (50) monostearate, octylphenoxypoly (ethyleneoxy) ethanol, decaglycerin penta-isostearate, sorbitan sesquioleate, hydroxylated lanolin, lanolin, triglyceryl diisostearate, polyoxyethylene (2) oleyl ether, calcium stearoyl-2-lactylate, methyl glucoside sesquistearate, sorbitan monopalmitate, methoxy polyethylene glycol-22/dodecyl glycol copolymer (Elfacos E200), polyethylene glycol-45/dodecyl glycol copolymer (Elfacos ST9), polyethylene glycol 400 distearate, and lanolin derived sterol extracts, glycol stearate and glycerol stearate; alcohols, such as cetyl alcohol and lanolin alcohol; myristates, such as isopropyl myristate; cetyl palmitate; cholesterol; stearic acid; propylene glycol; glycerin, sorbitol and the like.

The compounds or compositions described herein can be used alone or in combination with other active agents. In some embodiments, the active agent of the composition provided herein consists of a compound having Formula I-IV.

Typical compositions of compounds of Formula I-IV are topical gel compositions comprising the following inactive ingredients: Polyethylene glycol 400. Transcutol (i.e., diethylene glycol monoethyl ether), isopropyl alcohol, diisopropyl adipate, benzyl alcohol, Carbopol 980 (i.e., carbomer homopolymer type C), sodium hydroxide and purified water.

Several typical examples of such compositions are provided in Examples 1 and 2.

### Methods

### Formulation of a Topical Compositions Comprising a Compound of Formula IV or Formula I

The LUT014 (Formula IV) drug product (Example 1) is formulated as an aqueous-based gel containing organic solvents in which the LUT014 drug substance is in solution. The gel is yellow to green-yellow, clear, slightly viscous, and easily spread. The pH ranges between 5 and 7.

The inactive ingredients of the LUT014 Gel drug product are as follows: polyethylene glycol 400, Transcutol (i.e., diethylene glycol monoethyl ether), isopropyl alcohol, diisopropyl adipate, benzyl alcohol, Carbopol 980 (i.e., carbomer homopolymer type C), sodium hydroxide, and purified water.

The LUT017 (one of the Formula I family of compounds) is similarly formulated and administered. (Example 2).

### Administration of the Topical Compositions Comprising a Compound of Formula I-IV

### Target Populations:

### 1. Cutaneous Histiocytosis Subjects Having Only Histiocytosis Skin Lesions.

These subjects are treated with a single topical composition comprising at least one compound of Formula I-IV of this disclosure.

In some embodiments, the method of administration of the target population 1 provides treatment of cutaneous histiocytosis by topical administration of a composition comprising a therapeutically effective amount of at least one compound of Formula I-IV on the cutaneous lesions of a histiocytosis subject in need thereof.

### 2. Histiocytosis Subjects Having Also Histiocytosis Cutaneous Lesions (Usually Under the Eyes)

The subjects of population 2 are treated concomitantly with a topical composition comprising a therapeutically effective amount of at least one compound of Formula I-IV of this disclosure and with an approvable oral treatment selected from the following:
a. Oral vemurafenib
b. Any other regulatory approvable BRaf inhibitor effective for systemic histiocytosis treatment
c. Any other regulatory approvable treatment effective for systemic histiocytosis treatment

In some aspects, this disclosure provides a method for treatment of histiocytosis accompanied with topical lesions, by concomitant administration of two drugs or treatments to a subject in need thereof, having histiocytosis accompanied with cutaneous lesions:
1. A topical composition comprising at least one compound of Formula I-IV administered to the skin of said subject.
2. An oral approvable oral treatment selected from the following:
   a. Oral vemurafenib
   b. Any other regulatory approvable BRaf inhibitor effective for systemic histiocytosis treatment
   c. Any other regulatory approvable treatment effective for systemic histiocytosis treatment

In one embodiment, provided herein is a method of treatment, prevention of histiocytosis escalation and reducing severity of the histiocytosis in a subject in need thereof, wherein the treatment includes administration of a topical composition comprising a therapeutically-effective amount of a compound of Formula (I), Formula (II), Formula (III) or Formula (IV), a pharmaceutically acceptable salt or a solvate thereof, or a combination thereof and a pharmaceutically acceptable carrier or excipient and a therapeutically effective amount of oral vemurafenib (e.g. Zelboraf™) or any other approvable therapeutically effective drug or treatment.

In one embodiment, there is provided a method of treatment, prevention of histiocytosis escalation, reducing the histiocytosis severity wherein the treatment includes administration of a topical composition comprising a therapeutically effective amount of a compound of Formula I-IV and a pharmaceutically acceptable carrier or excipient.

In some embodiments, the method reduces the severity or prevents escalation of the histiocytosis cutaneous lesions.

In some embodiments, the topical composition comprises about 0.01% to 0.30% weight/weight of the compound of Formula I-IV, the method reduces the severity of the histiocytosis accompanied with topical lesions.

In some embodiments, the therapeutically effective amount of vemurafenib is 960 mg oral vemurafenib twice daily or a lower or higher dose, according to doctor's orders.

In some embodiments, this disclosure relates to a topical pharmaceutical composition comprising from 0.01% weight/weight to 0.3% weight/weight of a compound of Formula IV and a pharmaceutically acceptable carrier or excipient.

In some embodiments, the composition is in the form of a gel, a hydrogel, an ointment, a cream, a foam, a spray, a lotion, a liquid or a dermal patch.

In some embodiments, the topical composition comprises from 0.03% to 0.25% weight/weight of the compound of Formula IV.

In some embodiments, the topical composition comprises about 0.01% to 0.30% weight/weight of the compound of Formula IV.

In some embodiments, the topical composition comprises about 0.01% to 0.1% weight/weight of the compound of Formula IV.

In some embodiments, the topical composition comprises about 0.01% to 1% 20 weight/weight of a compound of Formula I-IV.

In some embodiments, the subject is a mammal, human or non-human. In some embodiments, the subject is a human.

In some embodiments, methods disclosed herein comprise topical administration of a therapeutically effective amount of the compounds/compositions of the present disclosure.

Methods comprising topical administration comprise local administration of any of the compositions disclosed herein to the skin of the subject. In some embodiments, topical administration comprises topically administering a composition, wherein the composition is a gel, a hydrogel, an ointment, a cream, a spray, a dermal patch, a paste, a foam, a lotion or a liquid.

In some embodiments, the amount of the compound of this disclosure administered depends on the nature of the compound, the formulation, and/or the severity of the cutaneous lesions. The therapeutically effective amount that is administered to a patient can be determined by dose-ranging clinical studies known in the art.

In some embodiments, the dosage regimen and/or length of treatment depends on the nature of the compound, the formulation, and/or the severity of the cutaneous lesions.

According to some embodiments, the method comprises topically administering the pharmaceutical composition comprising at least one compound of Formula I-IV once daily for 4 to 6 weeks, to the skin of the histiocytosis subject in need thereof.

In some embodiments, the method comprises topically administering the topical pharmaceutical composition once daily, to a skin lesion area affected by cutaneous histiocytosis.

In some embodiments, the topical pharmaceutical composition is administered once daily for 2 weeks, 3 weeks, 4 weeks, 5 weeks, 6 weeks, etc. In some embodiments, the topical composition is administered once daily for 4 to 6 weeks.

In some embodiments, the topical pharmaceutical composition is administered twice daily for 2 weeks, 3 weeks, 4 weeks, 5 weeks, 6 weeks, etc. In some embodiments, the topical pharmaceutical composition is administered twice daily for 4 to 6 weeks.

In some embodiments, the method comprises topically administering, once daily, to a skin area affected by the cutaneous lesions the topical pharmaceutical composition comprising from about 0.01% to 1% w/w, from 0.01% to 0.1% w/w, from 0.01% to 0.3% w/w, from 0.03% to 1% w/w, from 0.03% to 0.3% w/w, from 0.03% to 0.1% w/w, of the compound of Formula (IV) or a pharmaceutically acceptable salt or a solvate thereof, or a combination thereof, and a pharmaceutically acceptable carrier or excipient. In some embodiments, the topical pharmaceutical composition is administered once daily for 4 to 6 weeks.

In some embodiments, the method comprises topically administering, twice daily, to a skin area affected by the cutaneous lesions the topical pharmaceutical composition comprising from about 0.01% to 1% w/w, from 0.01% to 0.1% w/w, from 0.01% to 0.3% w/w, from 0.03% to 1% w/w, from 0.03% to 0.3% w/w, from 0.03% to 0.1% w/w of the compound of Formula IV or a pharmaceutically acceptable salt or a solvate thereof, or a combination thereof, and a pharmaceutically acceptable carrier or excipient. In some embodiments, the topical pharmaceutical composition is administered twice daily for 4 to 6 weeks.

In some embodiments, the administration results in a significant reduction in severity of the histiocytosis as early as 1 week after the initial administration of the topical pharmaceutical composition. In some embodiments, the methods reduce the severity of the cutaneous histiocytosis.

In some embodiments, the method provides sustained benefit for at least 7 days, 14 days, 21 days 28 days, 35 days, 42 days, 49 days, 56 days, or more after administration had terminated.

In some embodiments, the method prevents the apparition of cutaneous lesions when administered concomitantly with a second treatment.

In some embodiments, the methods disclosed herein reduce the severity of the cutaneous histiocytosis.

In some embodiments, success of treating cutaneous histiocytosis can be measured using methods known in the art.

In one embodiment, the methods disclosed herein prevent, partially or completely, the development of histiocytosis or cutaneous some embodiments, a method of treating and/or preventing escalation of histiocytosis lesions in a subject in need thereof is provided, the method comprising administering once a day for 4 to 6 weeks to the skin of the subject a topical pharmaceutical composition comprising from 0.01% weight/weight to 0.3% weight/weight of a compound of formula IV and a pharmaceutically acceptable carrier or excipient to the subject for treating or preventing histiocytosis lesions.

In some embodiments, the topical pharmaceutical composition is applied for four to six weeks to the skin lesions of the subject in need thereof.

In some embodiments, the topical composition comprises from 0.03% to 0.3% weight/weight of the compound of Formula IV.

In some embodiments, the topical composition comprises from 0.01% to 0.1% weight/weight of the compound of Formula IV.

In some embodiments, the topical composition comprises about 0.03% weight/weight of the compound of Formula IV.

In some embodiments, the topical pharmaceutical composition is in the form of a gel, a hydrogel, an ointment, a cream, a spray, a dermal patch, a foam, a lotion or a liquid.

In some embodiments, the topical pharmaceutical composition is in the form of a gel. In some embodiments, the topical pharmaceutical composition is in the form of a spray.

In some embodiments, the method reduces the severity or prevents escalation of the histiocytosis lesions. In some embodiments, the method reduces the severity of the histiocytosis lesions.

In some embodiments, compositions for use in the treatment and/or prevention of cutaneous lesions are provided, wherein the composition is topically administered to the skin of a subject in need thereof on a daily basis for 4 to 6 weeks, and the composition comprises from 0.01% to 1% w/w, from 0.01% to 0.1% w/w, from 0.01% to 0.3% w/w, from 0.03% to 1% w/w, from 0.03% to 0.3% w/w, from 0.01% to 0.1% w/w of the compound of Formula IV. In some embodiments the cutaneous lesions are histiocytosis lesions.

In some embodiments, compositions for use in the treatment and/or prevention of cutaneous lesions are provided, wherein the composition is topically administered to the skin of a subject in need thereof twice daily for 4 to 6 weeks, and the composition comprises from 0.01% to 1% w/w, from 0.01% to 0.1% w/w, from 0.01% to 0.3% w/w, from 0.03% to 1% w/w, from 0.03% to 0.3% w/w, from 0.03% to 0.25% w/w, from 0.03% to 0.1% w/w of the compound of Formula IV.

### EXAMPLES

The following examples illustrate certain embodiments of the disclosure but are not meant to limit the scope of the claims in any way. The following examples are put forth so as to provide those of ordinary skill in the art with a complete disclosure and description of how to make and use the described disclosure and are not intended to limit the scope of what the inventors regard as their disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1; Typical Gel Composition of the Compound of Formula IV (Code Name LUT014)

### Typical Gel Composition

The LUT014 drug product was formulated as an aqueous-based gel comprising LUT014, inactive ingredients and solvents in which the LUT014 drug substance is in solution. The gel is yellow to green-yellow, clear, slightly viscous, and easily spread. The pH ranges between 5 and 7.

The inactive ingredients and solvents of the LUT014 Gel drug product are as follows: polyethylene glycol 400, Transcutol (i.e., diethylene glycol monoethyl ether), isopropyl alcohol, diisopropyl adipate, benzyl alcohol, Carbopol 980 (i.e., carbomer homopolymer type C), sodium hydroxide, and purified water.

Three strengths of the compound of Formula IV (code name LUT014) were formulated for topical administration: 0.03% w/w, 0.10% w/w, and 0.25% w/w.

### LUT-014 (Formula IV, R=3-(trifluoromethoxy)phenyl) Formulation Ingredients of 0.03% w/w Gel Composition Comprising the Compound of Formula IV

LUT014 0.03% w/w
Polyethylene glycol 400, 20% w/w
Transcutol (i.e., diethylene glycol monoethyl ether), 20% w/w Isopropyl alcohol, 10% w/w
Diisopropyl adipate, 1.0% w/w Benzyl alcohol, 0.25% w/w
Carbopol 980 (i.e., carbomer homopolymer type C), 1% w/w Sodium hydroxide, to pH 6
Purified water q.s. to 100%.

### Prophetical:

### Example 2; Typical Gel Composition of a Compound of Formula I LUT017 (Formula I, R=4-chloro-3-(trifluoromethyl)phenyl) Formulation Ingredients of a Gel Composition Comprising a Compound of Formula I—(Code Name LUT017)

LUT017 0.015% w/w
Polyethylene glycol 400, 20% w/w
Transcutol (i.e., diethylene glycol monoethyl ether), 20% w/w
Isopropyl alcohol, 10% w/w
Diisopropyl adipate, 1.0% w/w
Benzyl alcohol, 0.25% w/w
Carbopol 980 (i.e., carbomer homopolymer type C), 1% w/w
Sodium hydroxide, to pH 6
Purified water q.s. to 100% w/w.

### Example 3: Inhibition of B-Raf (V599E) Mutated Enzyme by LUT014

### ATP Concentration: 120 μM

| | | Mean | Activity | | |
|---|---|---|---|---|---|
| | | (Counts - | (% | | |
| Sample | Counts | Blanks) | Control) | Mean | SD* |
| Lut014 / ET15060-42-P1 @ | 7996 | 7610 | 37 | 37 | 1 |
| 0.3 μM | 7831 | | 36 | | |
| CONTROL | 19150 | 20737 | 91 | 100 | 7 |
| | 22626 | | 108 | | |
| | 21655 | | 103 | | |
| | 20731 | | 99 | | |
| BLANK | 304 | / | / | / | / |
| | 304 | | / | | |

*NB. Where n = 2, the value reported here is actually range / √2

### Example 4: Administration of a Topical Composition Comprising a Compound of Formula I-IV (e.g. LUT014)

The LUT014 Gel is topically applied to the affected (or prone to be affected) skin area. A thin layer of the gel is be applied after washing and drying the treatment area.

Subjects are instructed to squeeze a small amount of the drug, up to about the size of three peas worth, depending on the size of the affected skin area) onto one or two fingertips (whichever is more convenient), dab it on the affected area of the chest, and spread the gel evenly over the entire surface of the affected area. The subjects are instructed to spread the gel by gently smoothing it into their skin and to avoid excessive rubbing.

Prophetical:

### Example 5: Topical Treatment, Prevention of Escalation and/or Reduction of the Severity of Histiocytosis in a Subject by Concomitant Administration of a First Composition Comprising a Therapeutically Effective Dose of at Least One Compound of Formula I-IV (e.g. Compound LUT014 of Formula IV) and a Therapeutically Effective Dose of Oral Vemurafenib

A subject having systemic histiocytosis and histiocytosis cutaneous lesions is treated concomitantly with a first topical composition comprising a therapeutically effective amount of at least one compound of Formula I_IV (e.g. LUT014 of Formula IV, 0.03% w/w)) and a second oral composition comprising a therapeutically effective dose of oral vemurafenib (e.g. Zelboraf™) 960 mg/day vemurafenib).

Any other in-between therapeutically effective amount detailed in the description of the instant disclosure may be useful.

The treatment includes topically administering once a day for 4 to 6 weeks to the skin of the subject in need thereof a topical pharmaceutical composition comprising from 0.01% weight/weight to 0.3% weight/weight (e.g. 0.03% w/w) of a compound of Formula I-IV and a pharmaceutically acceptable carrier or excipient to the subject for treating, preventing or reducing the severity of the histiocytosis lesions.

The administration of the oral vemurafenib and topical composition to a subject in need thereof is carried out concomitantly.

### Prophetical:

### Example 6: Topical Treatment, Prevention of Escalation and/or Reduction of the Severity of Histiocytosis Cutaneous Lesions by Administration to a Subject in Need Thereof of a Topical Composition Comprising a Therapeutically Effective Dose of at Least One Compound of Formula I-IV (e.g. Compound LUT014 of \Formula IV)

A subject having histiocytosis cutaneous lesions is treated with a topical composition comprising from 0.01% weight/weight to 0.3% weight/weight of a compound of Formula I-IV.

Any other therapeutically effective amount detailed in the description of the instant disclosure may be useful.

The treatment comprises topically administering once a day for 4 to 6 weeks to the skin of the subject in need thereof a topical pharmaceutical composition comprising from 0.01% weight/weight to 0.3% weight/weight of a compound of Formula I-IV and a pharmaceutically acceptable carrier or excipient to the subject for treating, preventing escalation or reducing the severity of the histiocytosis lesions.

### Example 7: LUT014 Drug Characterization

LUT014 has the chemical name of 5-N-(3-(9H-purin-6-yl)pyridin-2-yl)-6-methyl-1-N-(3-trifluoromethoxy)phenyl) isoquinoline-1,5diamine, with a molecular weight (MW) of 528.49 daltons (Da) and a molecular formula of C27H19F3N8O.

Three strengths of LUT014 formulated as an aqueous gel for topical administration were used in the clinical trial: 0.3 mg/g [0.03% weight/weight (w/w)], 1.0 mg/g (0.10% w/w), and 2.5 mg/g (0.25% w/w).

### Prophetical:

### Example 8: Topical Treatment, Prevention of Escalation and/or Reduction of the Severity of Histiocytosis in a Subject by Concomitant Administration of a First Composition Comprising a Therapeutically Effective Dose of LUT012 Compound

Subjects having histiocytosis cutaneous lesions are treated with a topical composition comprising from 0.01% weight/weight to 0.3% weight/weight of a compound LUT012. As a control, a placebo treatment (vehicle) is provided to control treated subjects.

The subjects are treated with the topical composition at least once daily for four to six weeks to the histiocytosis skin lesions of the subjects. Control subjects are treated with a corresponding suitable placebo composition, which does not include LUT012 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 9: Topical Treatment, Prevention of Escalation and/or Reduction of the Severity of Histiocytosis in a Subject by Concomitant Administration of a First Composition Comprising a Therapeutically Effective Dose of LUT013 Compound

Subjects having histiocytosis cutaneous lesions are treated with a topical composition comprising from 0.01% weight/weight to 0.3% weight/weight of a compound LUT013. As a control, a placebo treatment (vehicle) is provided to control treated subjects.

The subjects are treated with the topical composition at least once daily for four to six weeks to the histiocytosis skin lesions of the subjects. Control subjects are treated with a corresponding suitable placebo composition, which does not include LUT013 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 10: Topical Treatment, Prevention of Escalation and/or Reduction of the Severity of Histiocytosis in a Subject by Concomitant Administration of a First Composition Comprising a Therapeutically Effective Dose of LUT014 Compound

Subjects having histiocytosis cutaneous lesions are treated with a topical composition comprising from 0.01% weight/weight to 0.3% weight/weight of a compound LUT014. As a control, a placebo treatment (vehicle) is provided to control treated subjects.

The subjects are treated with the topical composition at least once daily for four to six weeks to the histiocytosis skin lesions of the subjects. Control subjects are treated with a corresponding suitable placebo composition, which does not include LUT014 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 11: Topical Treatment, Prevention of Escalation and/or Reduction of the Severity of Histiocytosis in a Subject by Concomitant Administration of a First Composition Comprising a Therapeutically Effective Dose of LUT015 Compound

Subjects having histiocytosis cutaneous lesions are treated with a topical composition comprising from 0.01% weight/weight to 0.3% weight/weight of a compound LUT015. As a control, a placebo treatment (vehicle) is provided to control treated subjects.

The subjects are treated with the topical composition at least once daily for four to six weeks to the histiocytosis skin lesions of the subjects. Control subjects are treated with a corresponding suitable placebo composition, which does not include LUT015 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 12: Topical Treatment, Prevention of Escalation and/or Reduction of the Severity of Histiocytosis in a Subject by Concomitant Administration of a First Composition Comprising a Therapeutically Effective Dose of LUT016 Compound

Subjects having histiocytosis cutaneous lesions are treated with a topical composition comprising from 0.01% weight/weight to 0.3% weight/weight of a compound LUT016. As a control, a placebo treatment (vehicle) is provided to control treated subjects.

The subjects are treated with the topical composition at least once daily for four to six weeks to the histiocytosis skin lesions of the subjects. Control subjects are treated with a corresponding suitable placebo composition, which does not include LUT016 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 13: Topical Treatment, Prevention of Escalation and/or Reduction of the Severity of Histiocytosis in a Subject by Concomitant Administration of a First Composition Comprising a Therapeutically Effective Dose of LUT017 Compound

Subjects having histiocytosis cutaneous lesions are treated with a topical composition comprising from 0.01% weight/weight to 0.3% weight/weight of a compound LUT017. As a control, a placebo treatment (vehicle) is provided to control treated subjects.

The subjects are treated with the topical composition at least once daily for four to six weeks to the histiocytosis skin lesions of the subjects. Control subjects are treated with a corresponding suitable placebo composition, which does not include LUT017 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 14: Treating Cutaneous Lesions in a Histiocytosis Subject with a Systemically Administered Composition Comprising a Therapeutically Effective Dose of LUT012 Compound

To treat cutaneous conditions induced or caused by histiocytosis, subjects that have been diagnosed with such cutaneous conditions (i.e., subjects that have histiocytosis) are systemically administered with a composition comprising LUT012, to treat said condition. As a control, a placebo treatment (vehicle) is provided to control treated subjects.

Briefly, the treatment protocol includes the following steps:
Subjects—Patients treated cutaneous lesions caused by histiocytosis.
Treatment—the subjects are treated for 6-20 weeks with a systemic composition comprising LUT012 compound in a suitable dosage form. The dosage form is an oral dosage form comprising 200-1000 micrograms of LUT012. Control—subjects are treated with a corresponding suitable placebo composition, which does not include LUT012 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 15: Treating Cutaneous Lesions in a Histiocytosis Subject with a Systemically Administered Composition Comprising a Therapeutically Effective Dose of LUT013 Compound

To treat cutaneous conditions induced or caused by histiocytosis, subjects that have been diagnosed with such cutaneous conditions (i.e., subjects that have bistiocytosis) are systemically administered with a composition comprising LUT013, to treat said condition. As a control, a placebo treatment (vehicle) is provided to control treated subjects.

Briefly, the treatment protocol includes the following steps:
Subjects—Patients treated cutaneous lesions caused by histiocytosis.
Treatment—the subjects are treated for 6-20 weeks with a systemic composition comprising LUT012 compound in a suitable dosage form. The dosage form is an oral dosage form comprising 200-1000 micrograms of LUT013. Control—subjects are treated with a corresponding suitable placebo composition, which does not include LUT013 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 16: Treating Cutaneous Lesions in a Histiocytosis Subject with a Systemically Administered Composition Comprising a Therapeutically Effective Dose of LUT014 Compound

To treat cutaneous conditions induced or caused by histiocytosis, subjects that have been diagnosed with such cutaneous conditions (i.e., subjects that have bistiocytosis) are systemically administered with a composition comprising LUT014, to treat said condition. As a control, a placebo treatment (vehicle) is provided to control treated subjects.

Briefly, the treatment protocol includes the following steps:
Subjects—Patients treated cutaneous lesions caused by histiocytosis.
Treatment—the subjects are treated for 6-20 weeks with a systemic composition comprising LUT012 compound in a suitable dosage form. The dosage form is an oral dosage form comprising 200-1000 micrograms of LUT014. Control—subjects are treated with a corresponding suitable placebo composition, which does not include LUT014 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 17: Treating Cutaneous Lesions in a Histiocytosis Subject with a Systemically Administered Composition Comprising a Therapeutically Effective Dose of LUT015 Compound

To treat cutaneous conditions induced or caused by histiocytosis, subjects that have been diagnosed with such cutaneous conditions (i.e., subjects that have bistiocytosis) are systemically administered with a composition comprising LUT015, to treat said condition. As a control, a placebo treatment (vehicle) is provided to control treated subjects.

Briefly, the treatment protocol includes the following steps:
Subjects—Patients treated cutaneous lesions caused by histiocytosis.
Treatment—the subjects are treated for 6-20 weeks with a systemic composition comprising LUT012 compound in a suitable dosage form. The dosage form is an oral dosage form comprising 200-1000 micrograms of LUT015. Control—subjects are treated with a corresponding suitable placebo composition, which does not include LUT015 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 18: Treating Cutaneous Lesions in a Histiocytosis Subject with a Systemically Administered Composition Comprising a Therapeutically Effective Dose of LUT016 Compound

To treat cutaneous conditions induced or caused by histiocytosis, subjects that have been diagnosed with such cutaneous conditions (i.e., subjects that have histiocytosis) are systemically administered with a composition comprising LUT016, to treat said condition. As a control, a placebo treatment (vehicle) is provided to control treated subjects.

Briefly, the treatment protocol includes the following steps:
Subjects—Patients treated cutaneous lesions caused by histiocytosis.
Treatment—the subjects are treated for 6-20 weeks with a systemic composition comprising LUT012 compound in a suitable dosage form. The dosage form is an oral dosage form comprising 200-1000 micrograms of LUT016. Control—subjects are treated with a corresponding suitable placebo composition, which does not include LUT016 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Prophetical:

### Example 19: Treating Cutaneous Lesions in a Histiocytosis Subject with a Systemically Administered Composition Comprising a Therapeutically Effective Dose of LUT017 Compound

To treat cutaneous conditions induced or caused by histiocytosis, subjects that have been diagnosed with such cutaneous conditions (i.e., subjects that have bistiocytosis) are systemically administered with a composition comprising LUT017, to treat said condition. As a control, a placebo treatment (vehicle) is provided to control treated subjects.

Briefly, the treatment protocol includes the following steps:
Subjects—Patients treated cutaneous lesions caused by histiocytosis.
Treatment—the subjects are treated for 6-20 weeks with a systemic composition comprising LUT012 compound in a suitable dosage form. The dosage form is an oral dosage form comprising 200-1000 micrograms of LUT017. Control—subjects are treated with a corresponding suitable placebo composition, which does not include LUT017 as an active substance.

During the treatment period, various parameters are tested, including, the rate of the cutaneous condition eruptions, the severity of the eruptions, the body location of the eruptions and other related cutaneous events. The body locations tested include face, neck and chest.

At the end of the trial treatment period, the tested parameters are determined by comparing between treated and placebo treated subjects, to determine treatment efficacy.

### Embodiments

In one embodiment, there is provided a method of treatment, prevention of escalation and/or reduction of the severity of histiocytosis in a subject in need thereof, comprising administration to the skin of a subject in need thereof a therapeutically effective amount of a topical pharmaceutical composition comprising a compound of Formula I,

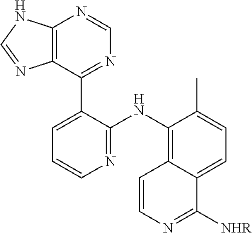

wherein R is selected from the group consisting of 3-ethynylphenyl, 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl, 3,5-dihydroxyphenyl or phenyl-3-sulfonamide, or a pharmaceutically acceptable salt or a solvate thereof, or a compound of Formula II

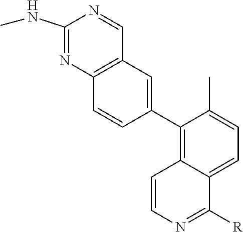

wherein R is NHR¹, wherein R¹ is 2-fluoro-4-iodophenyl, or a pharmaceutically acceptable salt or a solvate thereof, or a compound of Formula III

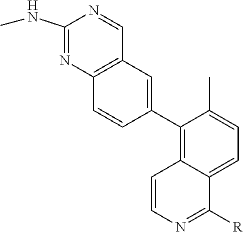

wherein R is NHR¹, wherein R¹ is 3-ethynylphenyl, 3-chloro-4-fluorophenyl, 2-fluoro-4-iodophenyl, or 4-chloro-3-(trifluoromethyl)phenyl, or a pharmaceutically acceptable salt or a solvate thereof, or a compound of Formula IV, which is the compound of Formula I, wherein R is —OCF3, (code name LUT014, Formula IV):

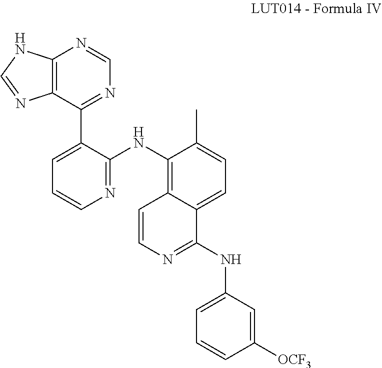 [SMILES: Cc1ccc2c(Nc3cccc(OC(F)(F)F)c3)nccc2c1Nc1ncccc1-c1ncnc2[nH]cnc12]

and a pharmaceutically acceptable carrier or excipient.

In one embodiment, there is provided a method of treatment, prevention of escalation and/or reduction of the severity of histiocytosis in a subject in need thereof, wherein the histiocytosis subject in need thereof has only cutaneous histiocytosis, wherein the treatment comprises administration to the skin of the subject a topical composition comprising a therapeutically effective dose of at least one compound of Formula I-IV.

In one embodiment, there is provided a method of treatment, prevention of escalation and/or reduction of the severity of histiocytosis in a subject in need thereof, wherein the histiocytosis subject has histiocytosis accompanied by cutaneous lesions, wherein the method comprises concomitant administration of a topical composition comprising a therapeutically effective dose of at least one compound of Formula I-IV and a therapeutically effective dose of a drug or treatment selected from: a. oral vemurafenib b. any other regulatory approvable BRaf inhibitor effective for systemic histiocytosis treatment or c. any other regulatory approvable treatment effective for systemic histiocytosis treatment.

In one embodiment, there is provided a method of treatment, prevention of escalation and/or reduction of the severity of histiocytosis in a subject in need thereof, wherein the histiocytosis subject has histiocytosis accompanied by cutaneous lesions, wherein the method comprises concomitant administration of a topical composition comprising a therapeutically effective dose of at least one compound of Formula I-IV and a therapeutically effective dose of an oral second treatment.

In one embodiment, there is provided a method of treatment, prevention of escalation or reduction of the severity of cutaneous lesions in a histiocytosis subject, comprising administration to the skin of the subject a therapeutically effective amount of a topical pharmaceutical composition comprising a compound of Formula I-IV, wherein the topical pharmaceutical composition is applied for four to six weeks to the histiocytosis skin lesions of the subject in need thereof.

In one embodiment, there is provided a method of treatment, prevention of escalation or reduction of the severity of cutaneous lesions in a histiocytosis subject, comprising administration to the skin of the subject a therapeutically effective amount of a topical pharmaceutical composition comprising a compound of Formula I-IV, wherein the topical pharmaceutical composition is in the form of a gel, a hydrogel, an ointment, a cream, a spray, a dermal patch, a foam, a lotion or a liquid.

In one embodiment, there is provided a method of treatment, prevention of escalation or reduction of the severity of cutaneous lesions in a histiocytosis subject in need thereof, comprising administration to the skin of the subject a therapeutically effective amount of a topical pharmaceutical composition comprising a therapeutically-effective amount of a compound of Formula I-IV, wherein the topical pharmaceutical composition is in the form of a gel.

In one embodiment, there is provided a method of treatment, prevention of escalation or reduction of the severity of cutaneous lesions in a histiocytosis subject in need thereof, comprising administration to the skin of the subject a therapeutically effective amount of a topical pharmaceutical composition comprising a compound of Formula I, II, III or IV, wherein the topical pharmaceutical composition is in the form of a spray.

In one embodiment, there is provided a method of treatment, prevention of escalation or reduction of the severity of cutaneous lesions in a histiocytosis subject in need thereof, comprising administration to the skin of the subject a therapeutically effective amount of a topical pharmaceutical composition comprising a compound of Formula I-IV, wherein the subject in need thereof has a histiocytosis disease.

In one embodiment, there is provided a method of treatment, prevention of escalation or reduction of the severity of cutaneous lesions in a histiocytosis subject in need thereof, comprising administration to the skin of the subject a therapeutically effective amount of a topical pharmaceutical composition comprising a compound of Formula I, II, III or IV, wherein the method reduces the severity or prevents escalation of the histiocytosis lesions.

In one embodiment, there is provided a topical pharmaceutical composition comprising from 0.01% to 1.00% weight/weight of the compound of Formula IV.

In one embodiment, there is provided a topical pharmaceutical composition comprising from 0.01% weight/weight to 0.3% weight/weight of a compound of Formula IV:

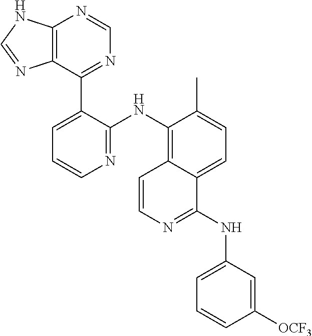 [SMILES: Cc1ccc2c(Nc3cccc(OC(F)(F)F)c3)nccc2c1Nc1ncccc1-c1ncnc2[nH]cnc12]

and a pharmaceutically acceptable carrier or excipient.

In one embodiment, there is provided a topical composition, comprising a therapeutically-effective amount of a compound of Formula I-IV, wherein the composition is in the form of a gel, a hydrogel, an ointment, a cream, a foam, a spray, a lotion, a liquid or a dermal patch.

In one embodiment, there is provided a topical composition, comprising a therapeutically-effective amount of a compound of Formula I-IV, wherein the composition comprising from 0.01 to 0.3% weight/weight of the compound of Formula IV.

In one embodiment, there is provided a topical composition, comprising a compound of Formula I-IV, wherein the composition comprising about 0.03% weight/weight of the compound of Formula IV.

In one embodiment, there are provided pharmaceutical topical compositions, comprising a therapeutically-effective amount of at least one compound of Formula I-IV and methods of using pharmaceutical topical compositions comprising a compound of Formula I-IV for the treatment, prevention of histiocytosis escalation and reduction of the severity of histiocytosis.

1. A method of treatment, prevention of escalation and/or reduction of the severity of histiocytosis in a subject in need thereof, comprising administration to the skin of a subject in need thereof a therapeutically effective amount of a topical pharmaceutical composition comprising
A) a compound of Formula I, 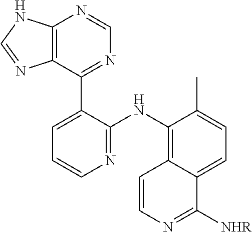
wherein R is selected from the group consisting of 3-ethynylphenyl, 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl, 3,5-dihydroxyphenyl or phenyl-3-sulfonamide, or a pharmaceutically acceptable salt or a solvate thereof; or
B) a compound of Formula II 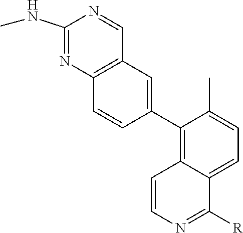
wherein R is NHR¹, wherein R¹ is 2-fluoro-4-iodophenyl, or a pharmaceutically acceptable salt or a solvate thereof; or
C) a compound of Formula III 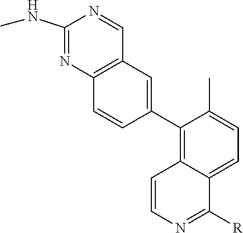
wherein R is NHR¹, wherein R¹ is 3-ethynylphenyl, 3-chloro-4-fluorophenyl, 2-fluoro-4-iodophenyl, or 4-chloro-3-(trifluoromethyl)phenyl, or a pharmaceutically acceptable salt or a solvate thereof; or
D) a compound of Formula IV, which is the compound of Formula I, wherein R is —OCF₃, (Formula IV): 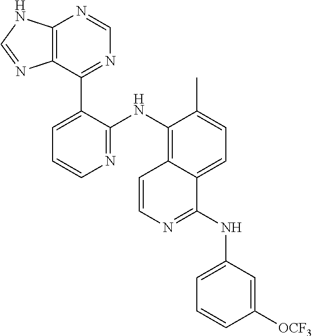 [SMILES: Cc1ccc2c(Nc3cccc(OC(F)(F)F)c3)nccc2c1Nc1ncccc1-c1ncnc2[nH]cnc12]
and a pharmaceutically acceptable carrier or excipient.

2. The method of claim 1, wherein the subject in need thereof has only cutaneous histiocytosis, wherein the method comprises administering to the subject in need thereof a topical composition comprising a therapeutically effective dose of at least one compound of Formula I-IV.

3. The method of claim 1, wherein the subject has histiocytosis accompanied by cutaneous lesions, wherein the method comprises treatment, prevention of escalation and/or reduction of the severity of histiocytosis in a subject by concomitant administration of a topical composition comprising a therapeutically effective dose of at least one compound of Formula I-IV, and a therapeutically effective dose of a drug or treatment selected from: oral vemurafenib, any other regulatory approvable BRaf inhibitor effective for systemic histiocytosis treatment, or any other regulatory approvable treatment effective for systemic histiocytosis treatment.

4. The method of claim 3, wherein the drug or treatment is vemurafenib.

5. The method of claim 1, wherein the topical pharmaceutical composition is applied once daily for four to six weeks to the histiocytosis skin lesions of the subject in need thereof.

6. The method of claim 1, wherein the topical pharmaceutical composition is in the form of a gel, a hydrogel, an ointment, a cream, a spray, a dermal patch, a foam, a lotion or a liquid.

7. The method of claim 1, wherein the topical pharmaceutical composition is in the form of a gel.

8. The method of claim 1, wherein the topical pharmaceutical composition is in the form of a spray.

9. The method of claim 1, wherein the subject in need thereof has a histiocytosis disease.

10. The method of claim 1, wherein the method treats, reduces the severity or prevents histiocytosis escalation.

11. The method of claim 1, wherein the topical pharmaceutical composition comprises from 0.01% to 1.0% weight/weight of the compound of Formula I-IV.

12. The method of claim 1, wherein the topical pharmaceutical composition comprises about 0.03% weight/weight of at least one of the compounds of Formula IV.

13. A topical pharmaceutical composition comprising from 0.01% weight/weight to 1.00% weight/weight of a compound of Formula I-IV and a pharmaceutically acceptable carrier or excipient.

14. The topical pharmaceutical composition of claim 13, wherein comprising from 0.01% weight/weight to 0.3% weight/weight of a compound of Formula IV and a pharmaceutically acceptable carrier or excipient.

15. The topical composition of claim 13, wherein the composition is in the form of a gel, a hydrogel, an ointment, a cream, a foam, a spray, a lotion, a liquid or a dermal patch.

16. The topical composition of claim 13, wherein the composition is in the form of a gel.

17. The topical composition of claim 13, wherein the composition is in the form of a spray.

18. The topical composition of claim 13, wherein comprising from 0.01 to 0.3% weight/weight of the compound of Formula IV.

19. The topical composition of claim 13, wherein comprising about 0.03% weight/weight of the compound of Formula IV.
